(19) United States
(12) Patent Application Publication
(10) Pub. No.: US 2009/0221867 A1
(43) Pub. Date: Sep. 3, 2009

(54) Integral Sling Connection System and Method

(75) Inventors: Jason Westrum Ogdahl, Robbinsdale, MN (US); Mona Nasseff Dahdah, West St. Paul, MN (US); Robert E. Lund, St. Michael, MN (US); Mark S. Bouchier, Lakeville, MN (US); Jose' W. Jimenez, Apple Valley, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/093,065

(22) PCT Filed: Oct. 25, 2006
(86) PCT No.: PCT/US06/60225

§ 371 Date: Sep. 10, 2008

Related U.S. Application Data
(60) Provisional application No. 60/735,364, filed on Nov. 11, 2005.
(60) Provisional application No. 60/795,348, filed on Apr. 27, 2006.
(60) Provisional application No. 60/841,479, filed on Aug. 31, 2006.

(51) Int. Cl. A61F 2/02; B29C 45/14

(52) U.S. Cl. 600/37; 264/257

(57) ABSTRACT

The present invention includes implantable sling systems for treating urinary incontinence and methods of forming such sling systems. In one embodiment, the sling includes connectors which are integrally molded over each end of the sling. The connectors are releasably attachable to a surgical needle during implantation in a tissue pathway to treat incontinence.

### RELATED APPLICATIONS

This application claims priority to PCT Application No. PCT/US2006/060225 filed Oct. 25, 2006, which claims the benefit of U.S. Provisional Application No. 60/735,364, filed Nov. 11, 2005; 60/795,348 filed Apr. 27, 2006; and 60/841,479 filed Aug. 31, 2006, all of which are incorporated herein in their entirety by reference.

### FIELD

The present invention pertains generally to surgically implantable mesh slings adapted to support a patient's urethra to treat urinary incontinence, to sling fabrication methods, and to sling implantation tools.

### BACKGROUND

Incontinence is a condition characterized by involuntary loss of urine or fecal matter beyond the individual's control, that results in the loss or diminution of the ability to maintain the urethral or fecal sphincter closed as the bladder or rectum fills with urine or fecal matter. Causes for this condition include damage and/or loss of support to the urethral sphincter, which can occur for a variety of reasons including pelvic accidents and aging related deterioration of muscle and connective tissue supporting the urethra.

One recognized method for treating incontinence is to implant a sling to support the urethra and then to secure respective sling ends in the retro pubic space surrounding the urethra. Elongated “self-fixating” or “tension-free” slings do not require physical attachment to tissue or bone, and instead rely on tissue ingrowth into sling pores to stabilize the sling. Such slings are reported for example, in commonly assigned U.S. Pat. Nos. 6,382,214, 6,641,524, 6,652,450, and 6,911,003 and publications and patents cited therein, each of which are hereby incorporated by reference in their entirety.

The implantation of tension-free urethral slings involve the use of implantation needles and other instruments that create transvaginal, transobturator, supra-pubic, or pre-pubic exposures or pathways adjacent the urethra. The needles further include a connection system for coupling the sling ends to the needle ends to draw sling end portions through the desired pathways.

As exemplified in FIG. 1, an implantable urethral sling 10 generally includes an elongate mesh 40 and detachable protective sheaths 26, 28 encasing the mesh at each end portion 22, 24 of the sling 10. Pre-formed dilators 12, 14 are attached to respective ends of the sheath using an adhesive, or by threading the sheath ends through an aperture in the dilators 12, 14 and then heat sealing the sheath ends.

The sling 10 is implanted in a patient using a surgical instrument such as the instruments 50 shown in FIGS. 2 and 3, which include handles 52 and needles 54. These needles 50 have opposing helical shapes that are designed to form right and left pathways through each obturator foramen between a lateral incision and an incision in a patient's vagina. Each instrument 50 is then associated with the dilators 12, 14. The dilators 12, 14 provide a relatively permanent attachment to the ends 62 of the needles 50. The end portions of the sling 10 are then drawn through the respective right and left obturator foramen (ROF/LOF) pathways of the pelvis (P) as shown in FIGS. 5 and 6 and further described in U.S. applications 2005/0043580 and 2005/0065395 publications and U.S. Pat. No. 6,911,003.

Generally speaking, the needle ends 62 are inserted axially into the dilators 12, 14, and the end portions 22, 24 of the urethral sling 10 are drawn through the pathways trailing the sling connectors 12, 14 and needles to draw a central support portion 30 against the urethra to treat urinary incontinence. The fixation of the needle ends 62 with the dilators 12, 14 is robust to inhibit inadvertent detachment as the connectors dilate the pathways and the end portions 22, 24 of the urethral sling 10 are drawn through the pathways. The connectors 12, 14 are drawn out through the skin incisions, and the urethral sling 10 and encasing sheaths 26, 28 are severed adjacent to the connectors 12, 14. The sheath portions 26, 28 and connectors 12, 14 are withdrawn from the right and left pathways over the sling mesh end portions 42, 44 exposing the urethral sling mesh to body tissue. The sling mesh end portions 42, 44 may be optionally sutured to subcutaneous tissue layers. Tissue pressure acutely stabilizes the exposed mesh, and tissue in-growth into the mesh pores chronically stabilizes the mesh in the pathway. Similar procedures for installing an elongated urethral sling to support the male urethra to alleviate incontinence are described in the above-referenced '450 patent, and the invention described herein is also suitable for treating male incontinence.

The needles disclosed in U.S. published application 2005/0043580 publication patent have a curvature in a single plane and correspond generally to the BioArc™ SP and SPARC™ single use sling needles sold by American Medical Systems, Inc. U.S. Pat. No. 6,911,003 describes needles having curvature in a three-dimensional space that may be used to advance and position a sling along transobturator pathways. These needles generally correspond to the Monarc™ needles sold by American Medical Systems.

Although the implantable slings and surgical needles described above function suitably, there is a need for slings having connections that easily attach to and detach from corresponding surgical needles. There is also a need for cost effective methods for manufacturing such slings.

### SUMMARY

One exemplary embodiment of the present invention provides a process for forming an implantable sling, which includes an elongate mesh material, by reducing the width of the first and second ends of the mesh and then molding a connector over each mesh end. In one embodiment, the molding process is carried out by injection molding. In another embodiment, the mesh ends are enclosed by one or more sheaths, and the connectors are molded over the sheaths.

Another embodiment of the present invention provides a system for treating urinary or fecal incontinence. The system includes a sling adapted to be implanted in a tissue pathway to treat urinary or fecal incontinence. The sling includes first and second sling connectors including a base portion injection molded over the first and second ends of the sling, and a filament loop secured to and extending from each base portion.

The system further includes first and second surgical instruments for implanting the sling. The first and second surgical instruments have a handle and respective right and left helical needle portions. The helical needle portions include a proximal needle end extending from the handle portion and a distal needle end including structure configured to releasably engage the sling connectors during implantation of the sling in the tissue pathway.

### BRIEF DESCRIPTION OF THE DRAWINGS

FIG. 1 shows a urethral sling which provides context for the incorporation of embodiments of the present invention.

FIG. 2 shows a surgical instrument for implanting a sling that includes a handle and a helical needle for insertion into a transobturator pathway of a patient.

FIG. 3 shows a surgical instrument for implanting a sling that includes a handle and a helical needle for insertion into a transobturator pathway of a patient.

FIG. 4 shows a connection system for associating a sling end with a surgical instrument.

FIG. 5 shows the surgical instrument of FIG. 3 extending along the left transobturator pathway.

FIG. 6 shows the surgical instrument of FIG. 2 extending along the right transobturator pathway.

FIG. 7 shows an implantable sling according to an embodiment of the present invention, which includes connectors having apertures for associating the sling with a surgical instrument.

FIG. 8 shows one end of the sling shown in FIG. 7, and the end of a surgical instrument having structure capable for associating the instrument with the sling.

FIG. 9 shows the sling end and needle end of FIG. 7 releasably associated as would occur during the implantation of the sling.

FIG. 10 shows a sling end according to another embodiment of the present invention, which includes a connector having a loop.

FIG. 11 shows a sling end having a loop connector, and a needle end releasably associated with the sling end as would occur during the implantation of the sling.

FIG. 12 shows a sling end according to another embodiment of the present invention, which includes a connector having a loop.

FIG. 13 shows a sling end according to another embodiment of the present invention, which includes a connector having a loop.

FIG. 14 shows a sling end according to another embodiment of the present invention, which includes a connector having a loop.

FIG. 15 shows a sling end according to another embodiment of the present invention, which includes a connector having a loop.

FIG. 16 shows a needle end according to another embodiment of the present invention, which includes an aperture for receiving a loop or similar sling connector.

FIG. 17 shows a sling connection system according to another embodiment of the present invention, which includes a sling connector having a loop and a needle end having a slot and recess for receiving the loop.

FIG. 18 shows a sling connection system according to another embodiment of the present invention.

FIG. 19 shows a sling connection system according to another embodiment of the present invention, which includes a sling connector having a loop with a reinforced portion and a needle end having a slot for receiving the loop.

FIG. 20 shows a needle end having an aperture, slot and recess for releasable association with a sling connector such as a loop connector.

FIG. 21 shows a needle end having multiple slots for receiving a sling connector such as a loop connector.

FIG. 22 shows multiple needle ends having various slot configurations for releasable association with a sling end connector such as a loop connector.

FIG. 23 shows a sling connection system according to another embodiment of the present invention.

FIG. 24 shows a sling implantation system or kit according to an embodiment of the present invention.

FIG. 25 shows the sling connection system of the implantation system shown in FIG. 24.

FIG. 26 show an end view of the surgical needle of the implantation system shown in FIG. 24.

FIG. 27 shows a cross-sectional view of the needle end of the implantation system shown in FIG. 24.

FIG. 28 shows a close-up view of the needle end of the implantation system shown in FIG. 24.

FIG. 29 shows a sling connection system according to another embodiment of the present invention, which includes a sling having a connector with a ball and cable structure, and a slot for releasably receiving the ball and cable.

FIG. 30 shows an alternate embodiment of the connector shown in FIG. 29.

FIG. 31 shows a top view of an alternate embodiment of the needle end shown in FIG. 29.

FIG. 32 shows a bottom view of the embodiment shown in FIG. 31.

FIG. 33 is a block diagram depicting the steps of forming insert molded sling connectors.

### DETAILED DESCRIPTION

In the following detailed description, references are made to illustrative embodiments of methods and apparatus for carrying out the invention. It is understood that other embodiments can be utilized without departing from the scope of the invention. Additionally, various aspects of the disclosed embodiments may be combined with aspects of other disclosed embodiments within the scope of the present invention. In particular, the connection systems described herein may be used with various slings, needles and implantation procedures in accordance with the present invention.

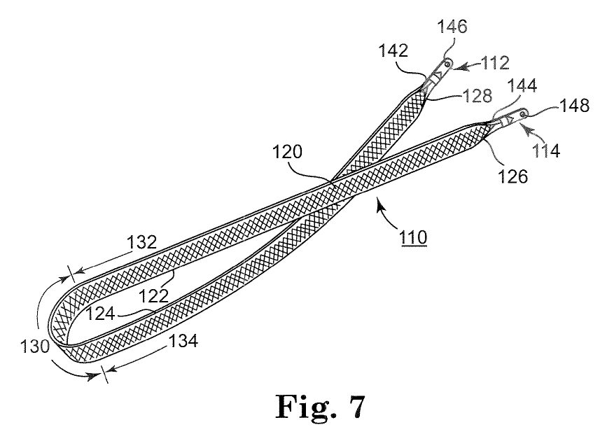

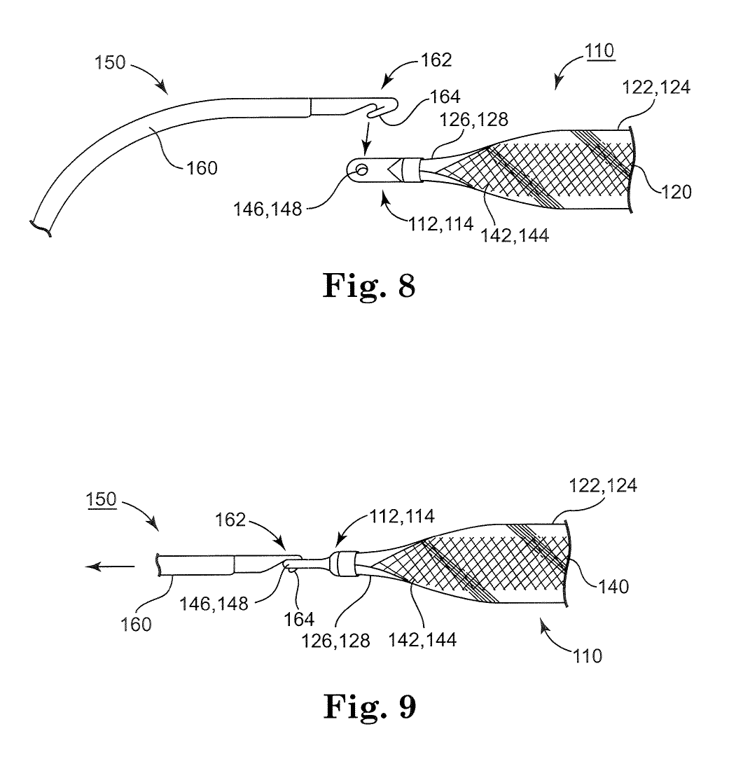

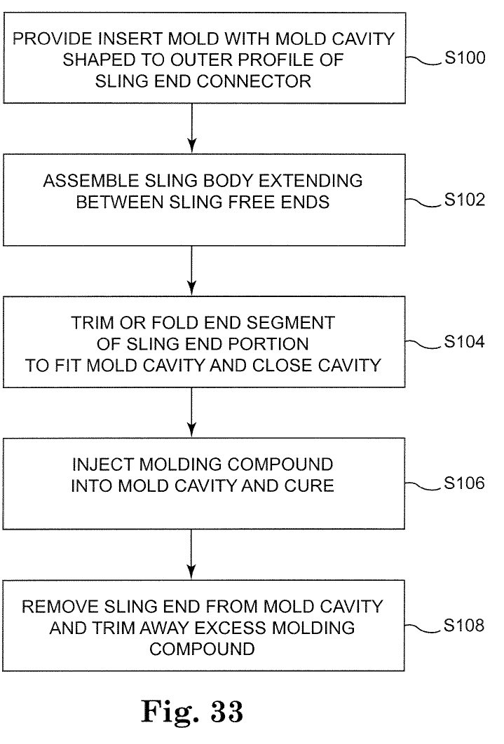

FIG. 7 illustrates an incontinence sling 110 for treating urinary or fecal incontinence according to one exemplary embodiment of the present invention. The sling 110 has sling connectors 112, 114 at respective end portions of the sling 110. The sling connectors 112, 114 include integrally molded loop portions 146, 148, which are readily attachable to and detachable from a surgical needle such as needle 150 illustrated in FIGS. 8-9. A sling body 120 further includes center portion 130 and end portions 132, 134 encased within sheaths 122, 124. Each of the sling connectors 112, 114 is molded over mesh ends 142, 144 and/or respective sheath ends 126, 128 by an injection molding process described below with reference to FIG. 33.

The center portion 130 and end portions 132, 134 can be formed from a synthetic material such as a polypropylene mesh. Alternatively some or all of these sling portions can be formed from a biocompatible material such as mammal dermis.

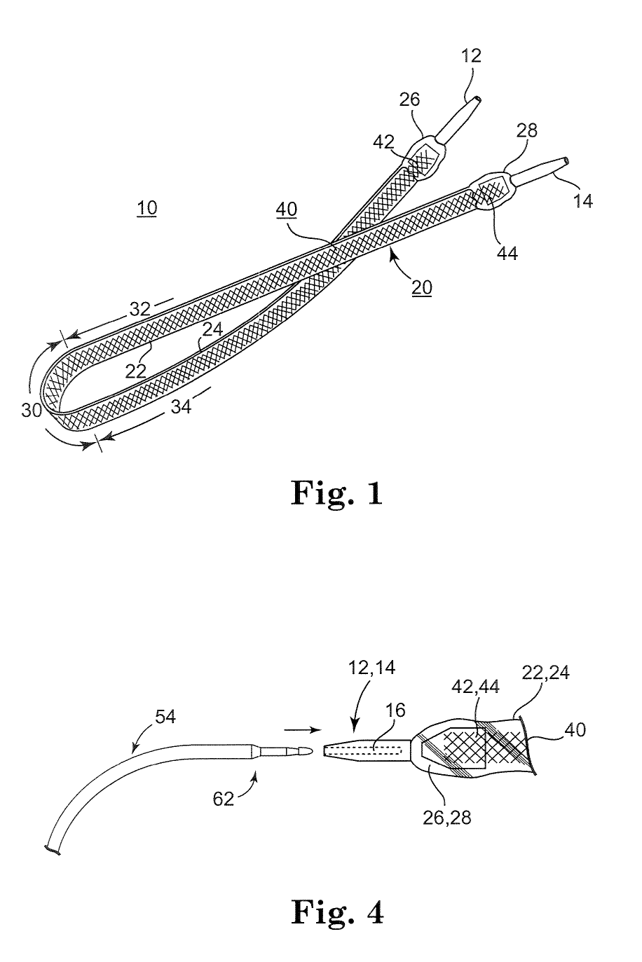

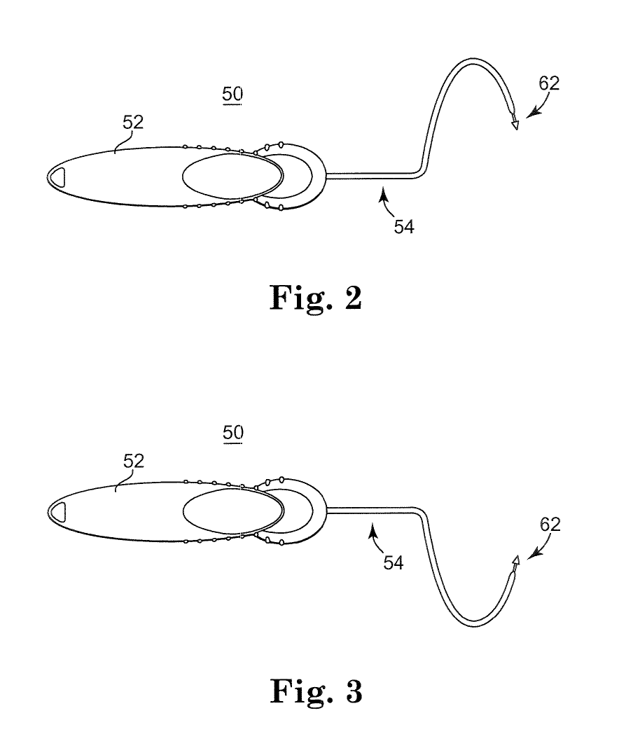

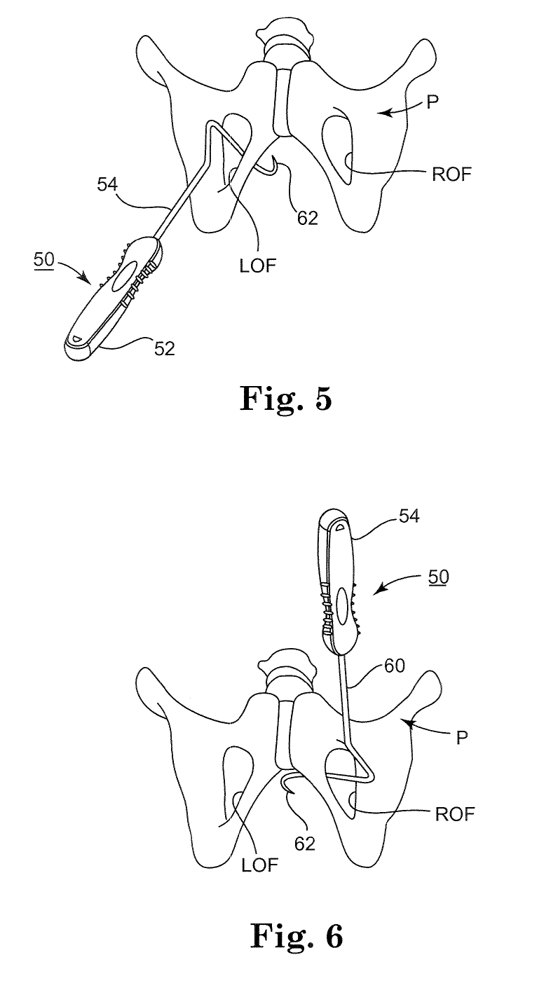

As further shown in FIGS. 8 and 9, the needle 150 includes a distal end 160 with a hook-shaped needle end 162. Suitable needle shapes 150 include the helical shapes shown in FIGS. 1-6, as well as other surgical needles suitable for implanting incontinence slings. The hook-shaped needle end 162 has a prong or hook 164 dimensioned to be received into the holes 146, 148 to draw the end portions 132, 134 (see FIG. 7) of the sling 110 through the tissue pathways formed by the needle 150 (see FIGS. 5 and 6). Additional suitable hook configurations described herein may also be used with the sling embodiments illustrated in FIGS. 7-9.

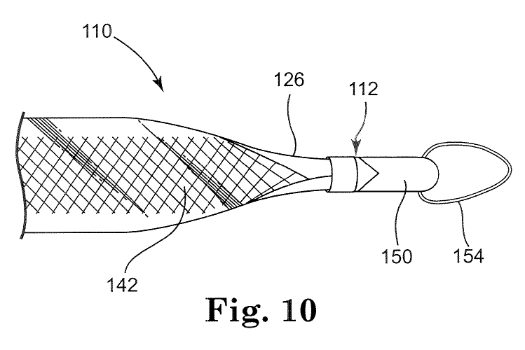

FIGS. 10-24 depict additional embodiments of the present invention, in which the sling connectors include a suture loop integrally formed with the sling sheath and/or mesh end. Suitable needles for use with this embodiment include the hook-shaped configuration described above and additional configurations described herein. It will be evident to persons of skill in the art that although only one sling end is shown and described, a second sling end having the same features is also present.

FIG. 10 illustrates a sling 110 end according to an embodiment of the present invention, which includes a sling connector 112 molded over the sling end 110, and more particularly over a sheath end 126 and/or a mesh end 142. The sling connector 112 includes a base portion 150 and a suture loop 154 secured to the base portion 150. The base portion 150 for this and other embodiments may be formed by injection molding a polymer material such as polypropylene or polyethylene over the sling end 110 as described in greater detail below. Suitable suture loops 154 or other filaments possess sufficient integrity to withstand the implantation process, while still having a significant degree of flexibility. Polyester sutures are suitable for certain embodiments due to its higher melting point. Other embodiments of the present invention include various natural and synthetic fibers and/or filaments, coated sutures (including reinforcement coatings), braided sutures or other suitable filament materials. The length of the suture loop 154 can vary depending on the type of sling being implanted, the configuration of the corresponding needle connector and the preference of the clinician(s) performing the procedure.

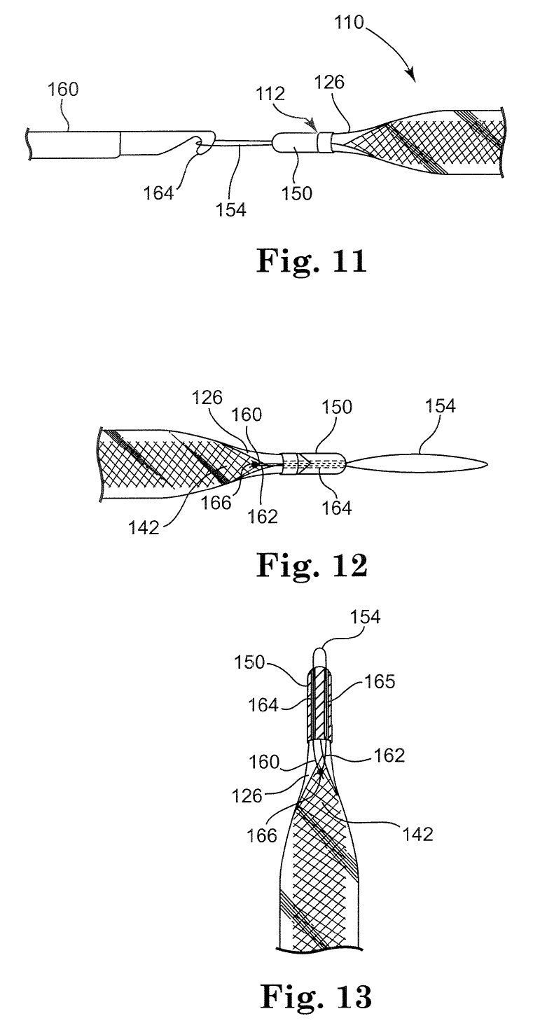

FIG. 11 shows a suture loop 154 releasably attached to hook 164 of needle 150 according to an embodiment of the present invention. In this manner, the sling 110 may be pulled through surgical pathways as described with reference to FIGS. 1-6 (or by other conventional implantation procedures), and the needle 150 can then be easily released or disengaged from the suture loop 154.

FIGS. 12-16 show various approaches for securing the suture loops 154 to the base portion 150 of the connector 112, to the sheath end 126 and/or to the mesh end 142. FIG. 12 shows a cross-sectional view of another embodiment of the present invention in which each end 160, 162 of a loop is inserted into a through-hole 164 in the base portion 150 and a knot 166 is then formed proximal to the base portion 150 to complete the suture loop 154 and to prevent the suture loop 154 from pulling through the base portion 150 during implantation. In this embodiment, the suture end 160 can be tied directly to the mesh end 142 or to the sheath end 126. If two sutures are used, another knot can be formed at the distal end of the sutures to complete the suture loop. The embodiment shown in FIG. 13 is similar to FIG. 12 except that the suture ends 160, 162 are each inserted through separate through-holes 164, 165.

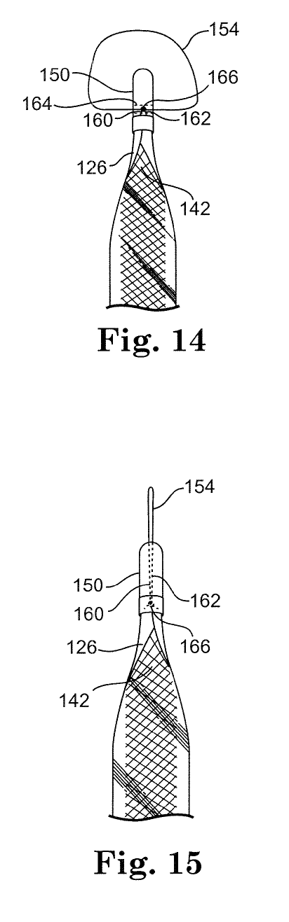

FIGS. 14 and 15 illustrate embodiments in which the suture loop 154 is secured to the base portion 150 during a molding process such as the injection molding process described with reference to FIG. 33. In the embodiment shown in FIG. 14, the through-hole 164 extends transversely to the length of the base portions 150.

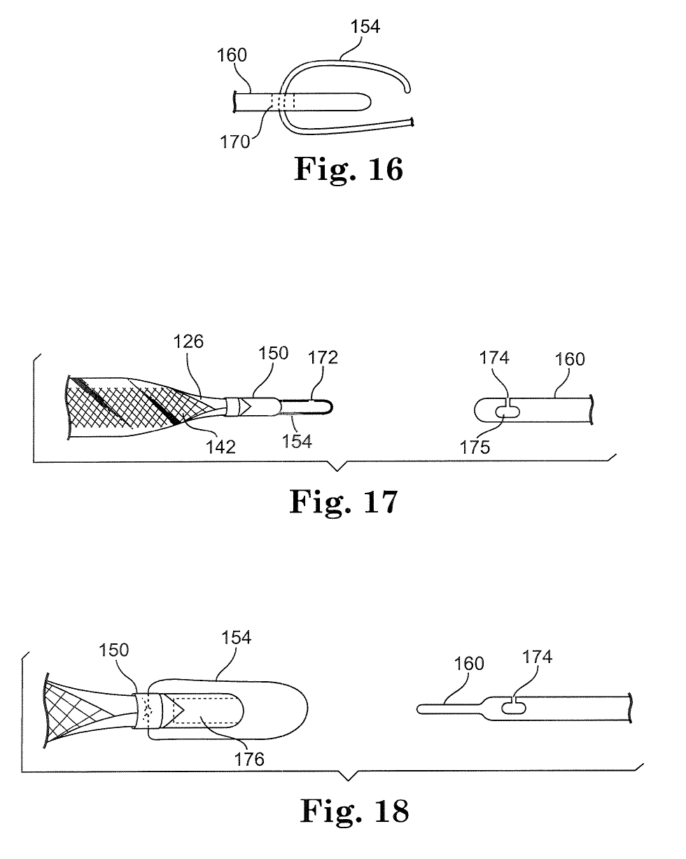

FIGS. 16-23 show additional embodiments of the present invention that utilize slings having suture loops at each end and connectors that releasably attach to the suture loops. FIG. 16 shows an embodiment in which a needle end 160 includes a through-hole 170, and the suture loop 154 is releasably associated with the needle end 160 by threading the suture loop 154 through the through-hole 170 and then hooking the loop 154 around the needle end 160. Alternatively, the needle end 160 could include both a through-hole 170 and a hook such that the suture loop 154 is first threaded through the through-hole 170, and is then further retained by the hook.

FIG. 17 shows an embodiment in which the suture loop 154 has a narrowed portion 172 which fits into a corresponding slot 174 and recess 175 in the needle end 160 such that the loop 154 is retained in the recess 175 during implantation unless the narrowed portion 172 is aligned with the slot 174.

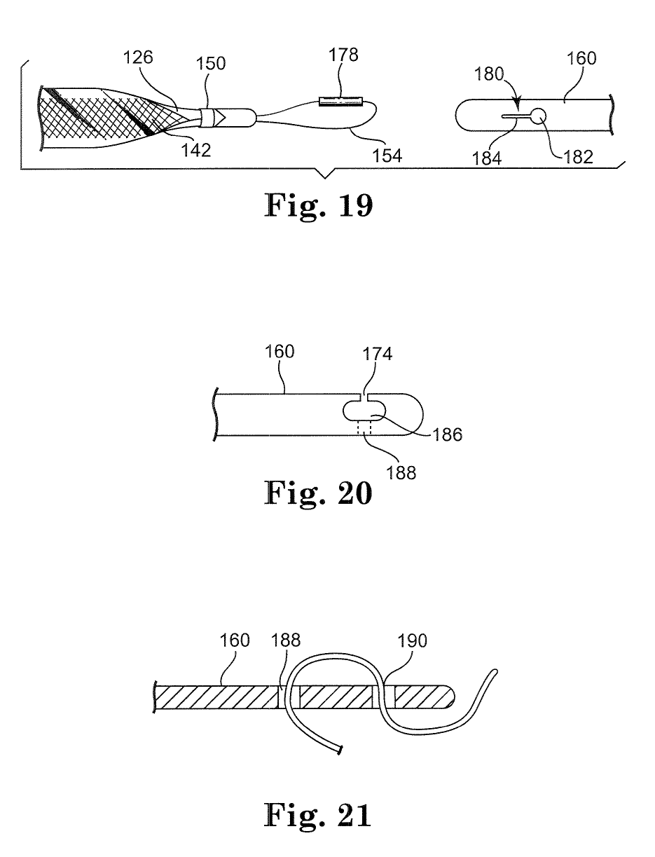

FIG. 18 shows an embodiment in which the base portion 150 includes a tube 176 into which the needle end 160 can be inserted in order to align the base portion 150 with the needle end 160 during implantation. FIG. 19 shows an embodiment in which a portion of the suture loop 154 includes a stiffener or bead 178, which can be inserted and releasably retained in a corresponding slot arrangement 180 that includes an opening 182 for receiving the bead 178 and a narrow slot 184 that retains the bead 178.

FIG. 20 shows a needle-hook embodiment having a slot 174, a recess 186 and a through-hole 188. In this embodiment, a suture loop can be threaded through the slot 174 and the through-hole 188 and is then looped back over the needle end 160 and into the slot 174 such that it is retained in the recess 186. This arrangement may reduce the chance that the loop unintentionally detaches from the needle end 160.

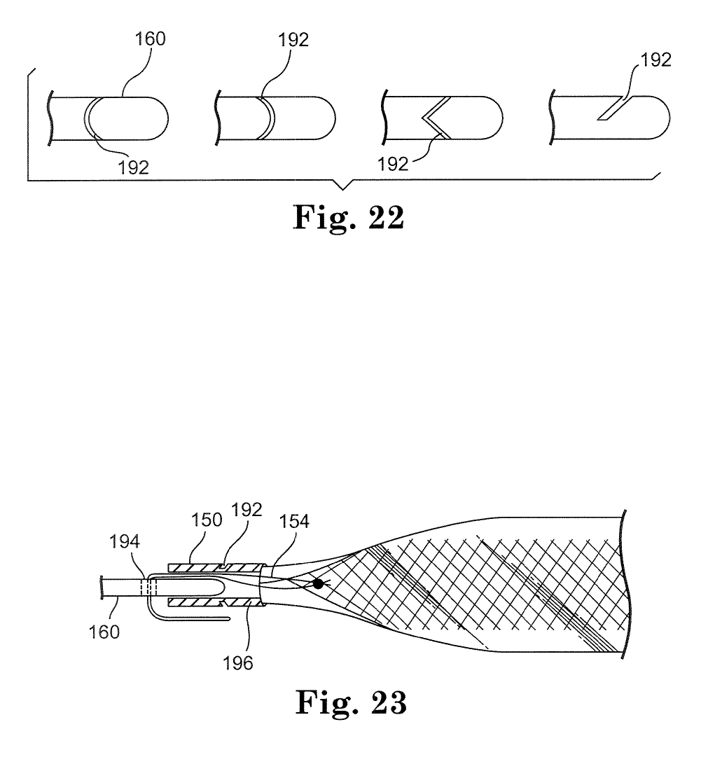

FIG. 21 shows an embodiment in which the needle end 160 includes multiple (2 or more) holes 188 and 190 through which a suture loop can be threaded prior to retaining the loop over the needle end 160. The needle end 160 may optionally include a hook or similar retention structure as well. FIG. 22 shows additional needle end 160 structure which include various slots 192 for retaining a suture loop.

FIG. 23 shows an embodiment in which the suture loop 154 is inserted through a through-hole 194 in the needle end 160, and is then hooked onto the base portion 150 of the sling connector instead of the needle end 160 as shown in previous embodiments. The base portion 150 in the illustrated embodiment includes a tube portion 196 to assist with aligning the needle end 160. The base portion 150 could also include a recess, slot, notch or other structure to further retain the loop during sling implantation.

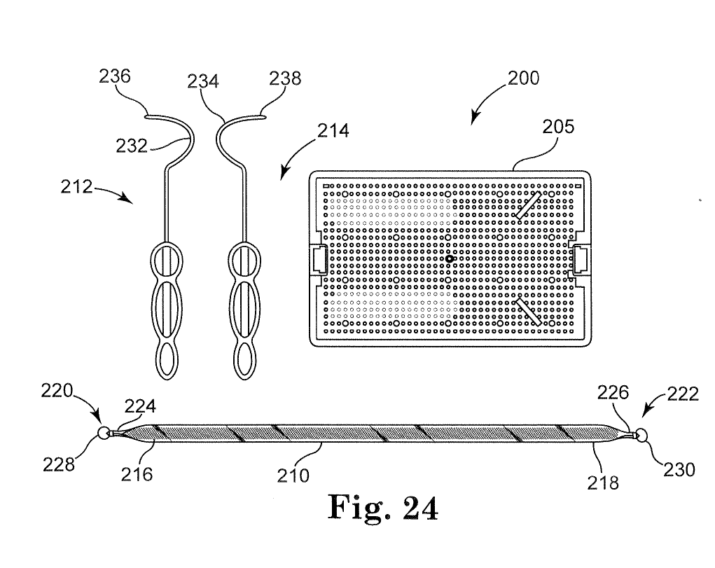

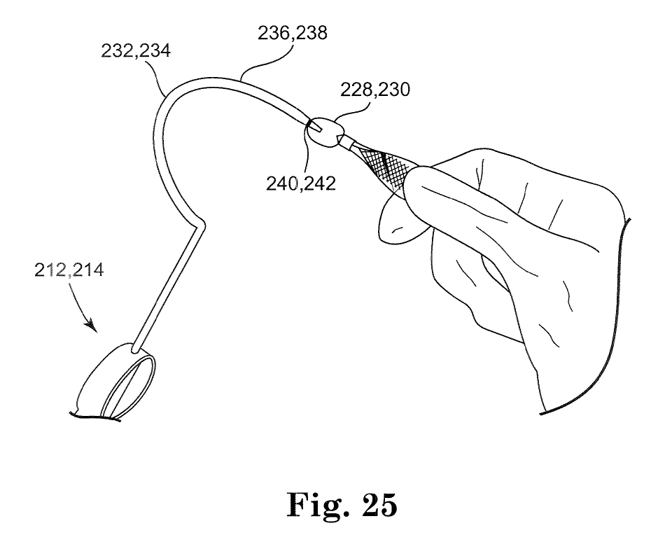

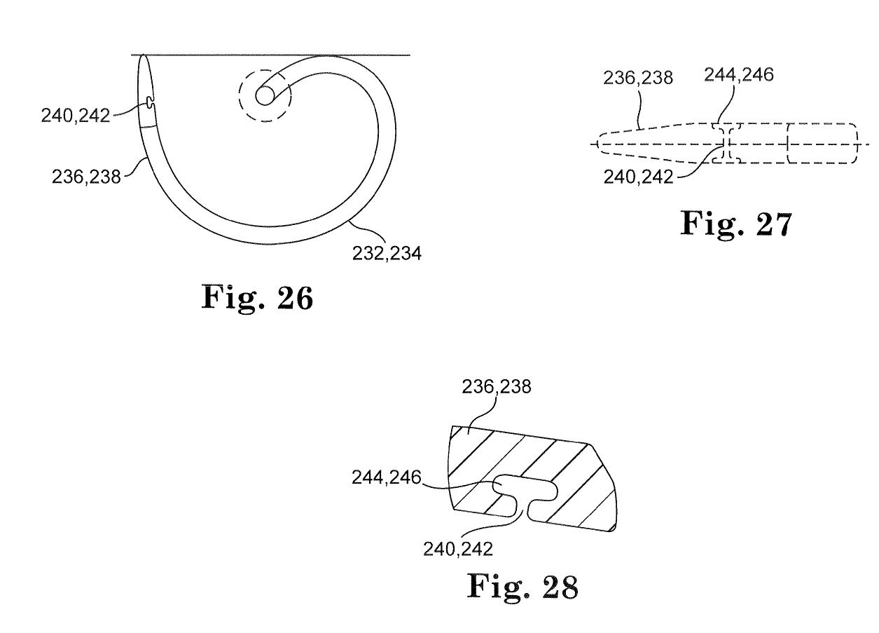

FIG. 24 shows an sling implantation assembly 200 according to an embodiment of the present invention. The assembly 200 includes a container 205, a sling 210, and surgical needles 212, 214. The sling 210 includes sling ends 216, 218 with connectors 220, 222. The connectors 220, 222 include respective base portions 224, 226 and loops 228, 230. The needles 212, 214 have opposing helixes 232, 234 for creating tissue pathways through the right obturator foramen and left obturator foramen of a patient, and needle ends 236, 238 for releasable association with the sling connectors 220, 222. As further shown in FIGS. 25-28, the needle ends 236, 238 include slots 240, 242 for receiving loops 228, 230, and recesses 244, 246 for releasably retaining the loops 228, 230 during implantation. From the perspective shown in FIG. 27, the slots 240, 242 and recesses 244, 246 form “H” shaped connectors.

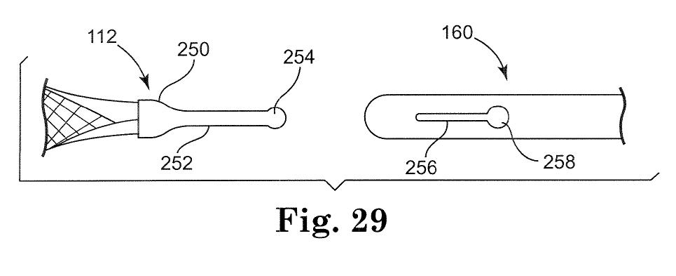

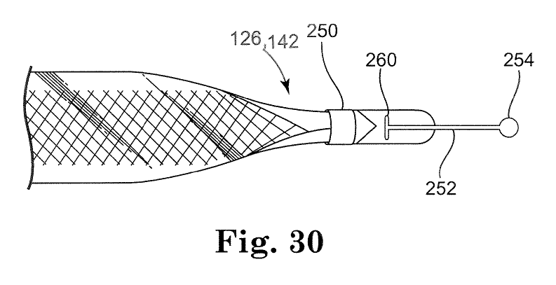

FIG. 29 illustrates an additional embodiment of the present invention, in which each sling end includes a connector 112 having a base portion 250, a cable 252 extending from base portion 250 and a ball portion 254 formed at a distal end of the cable 252. A corresponding needle end 160 includes a slot 256 with an opening 258 at one end. In use, the ball portion 254 can be inserted into the opening 258 and the cable 252 can then be pulled through the slot 256 to provide releasable association.

FIG. 30 illustrates an embodiment in which the base portion 250 is formed from a separate material than the cable 252 and ball 254 portions. In these embodiments, the base portion 250 may be molded over the sling end 142, 126 and 144 and 128 and around the cable 252 and ball 254 portions as described with reference to FIG. 33. One end of the cable 252 may also include an anchor 260 to retain the cable in the base portions 250. The cable 252 and ball 254 portions may be formed from the same type of material as the base portion 250. Alternatively, the base portion 250 could be formed from a polymer, and the cable 252 and ball 254 portions could be formed from a metal or a different polymer.

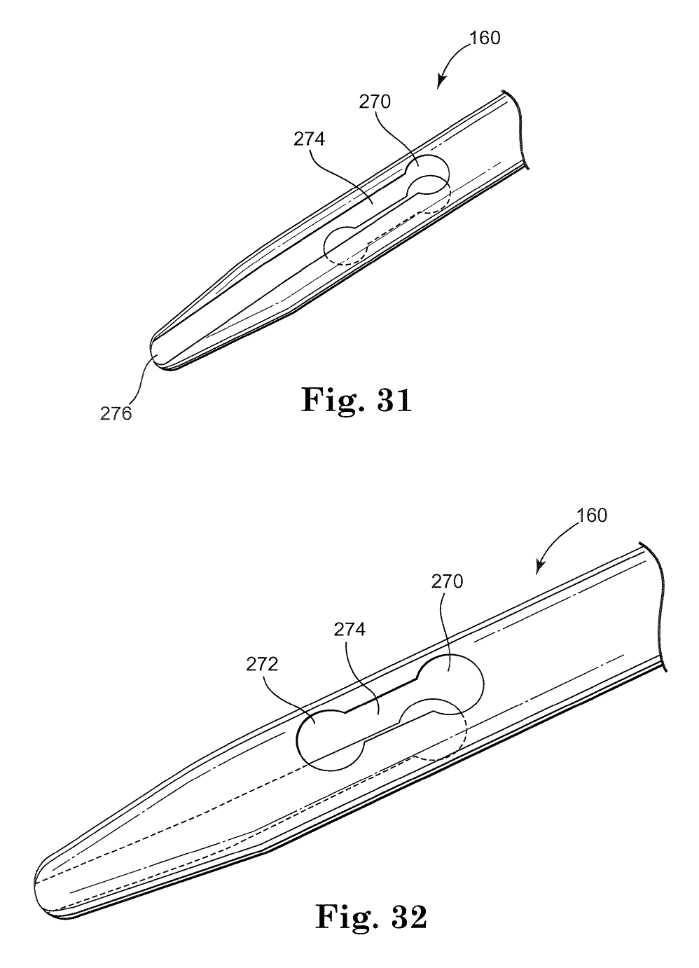

FIGS. 31 and 32 show various views of a needle end 160 having a slot and opening arrangement according to another embodiment of the present invention. As illustrated, the needle end includes a through-hole 270, a seat 272 located on one side of the needle, and a channel 274 extending therebetween. On the side of the needle end 160 opposite the seat 272, the channel 274 extends to a distal tip 276 of the needle end 160. In this embodiment, through-hole 270 is sized to receive the ball portion 254 of the sling connector (see FIG. 29) and to allow the ball portion 254 to extend through both sides of the through-hole 270. The channel 274 is configured to allow the cable portion 252 to pass through until the ball portion 254 is received by the recess 272. The seat 272 is sized such that the ball portion 254 is retained in the seat 272. Once the ball portion 254 is retained in the seat 272, the cable 252 can be aligned in the channel 274 up to the distal tip 276. The sling connector can be released from the needle end 160 by sliding the cable 252 in the opposite direction in the channel and then by extracting the ball portion 254 through the through-hole 270.

The needle embodiments shown in FIGS. 31 and 32 could be modified such that either side of the slot arrangement could retain the ball and cable by including a recess and groove on both sides of the needle. Additionally, although the foregoing embodiments illustrate a ball portion at the end of the cable, other shapes, including a half circle, cone cylinder, or disk could also be used. A slot portion could also be molded onto each sling end, and the ball and cable could be attached to the end of the needle in another embodiment.

The steps of forming and integrally attaching the sling connectors to the sheath and/or mesh ends for select embodiments is described in FIG. 33. In step S100, a suitable injection mold is provided that has a mold cavity shaped to receive an end portion of the sling body and to integrally form a base portion over each sling end. The sling is formed in step S102 following any suitable fabrication process that forms an elongate mesh sling that is generally contained within a protective sheath. End segments of the resulting sling are trimmed, longitudinally folded or otherwise formed into a generally conical configuration in step S104 to fit into a portion of the mold cavity. A molding compound including a polymer such as polypropylene or a derivative thereof is injected into the mold cavity and cured in step S106. The sling connector is removed from the mold and trimmed after curing in step S108.

In embodiments in which the base portion and other portions of the connector (e.g., suture loop) are formed from discrete components, the discrete connection components can be separately formed and then inserted into the mold cavity prior to molding the base portion over the sling ends. In this manner, the discrete connection component is integrally formed with the base portion. In other embodiments, the discrete connection components are attached to the base portion and/or sling ends after the base portion is molded over the sling ends via through-holes, interference fits and the like.

There are several potential benefits to securing the sling ends and connectors in this manner. For example, injection molding tends to be more efficient because a separate dilator need not be formed. Also, injection molding may be easier to automate on a commercial scale. Further, injection molding allows discrete connection components such as a suture loop to be secured to the sling end in a single step.

Various alternative embodiments are also contemplated as part of the present invention. For example, the mesh portions of the sling described herein could include a bioactive material at the central portion. Additionally, the central portion could be formed with an increased width compared to the sling ends to accommodate fecal incontinence treatment by implanting the sling via suprapubic, retropubic, transvaginal, transurethral, transobturator or near-obturator pathways. In further embodiments, the sling ends described herein could be replaced with tissue anchors that can be easily associated with surgical needle and fixed in hard or soft tissue via at least one vaginal or perineal incision.

Although embodiments of the present invention have been described with reference to the treatment of female urinary continence, it should be appreciated that many of these embodiments would also be suitable to implant and repair a variety of pelvic conditions in both males and females. For example, embodiments of the present invention would be suitable for a variety of pelvic floor repairs and/or treatments, including pelvic organ prolapse repair, levator hiatus repair, fecal incontinence treatment, male urinary incontinence treatment, perineal body support and hysterectomy support.

All patents, applications, and publications referenced herein are hereby incorporated by reference in their entireties.

1. A process for forming an implantable sling comprising:
providing an elongate rectangular mesh sized and shaped to be secured in a tissue pathway of a patient to treat incontinence, the mesh having first and second ends and a middle portion extending therebetween;
reducing the width of the first and second ends relative to the middle portion of the sling; and
molding a first connector around the first end and a second connector around the second end to secure the first and second connectors to the first and second ends.

2. The method of claim 1 wherein the method further comprises enclosing the first and second mesh ends in a sheath and wherein the molding step comprising molding the first and second connectors around the sheath.

3. The method of claim 2 wherein the enclosing step includes enclosing the first sling end with a first sheath and the second sling end with a second sheath.

4. The method of claim 3 wherein the first and second sheaths overlap at the middle portion of the elongate mesh.

5. The method of claim 1 wherein the molding step comprises injection molding the first and second connectors over the first and second ends.

6. The method of claim 5 wherein the injection molding step includes the step of inserting each sling end into a mold and forming the first and second connectors over the sling ends.

7. The method of claim 1 wherein the connector includes a base portion and a discrete loop portion, and wherein the molding step includes forming the base portion over the sling end and the discrete loop portion to form the connector.

8. The method of claim 1 wherein the molding step includes the step of injection molding a unitary connector including a base portion molded over the sling end, a cable portion extending from the base portion, and a ball portion at a distal end of the cable portion.

9. The method of claim 1 wherein the forming step includes the step of injection molding a unitary connector including a base portion and a loop portion formed from the same material as the base portion.

10. A sling implantation system including:
a sling adapted to be implanted in a tissue pathway to treat incontinence, the sling including an elongate mesh body having first and second ends;
first and second sling connectors associated with the first and second ends, the sling connectors including a base portion molded over each end of the sling; and
at least one instrument for implanting the sling that includes a handle and a needle, the needle including a proximal portion extending from the handle, a curved portion adjacent the proximal portion and a distal portion that includes a structure configured to releasably engage the first or second sling connectors during implantation of the sling in the tissue pathway.

11. The system of claim 10 wherein the first and second connectors include a loop portion extending from the base portion.

12. The system of claim 11 wherein the loop portion comprises a filament loop portion.

13. The system of claim 12 wherein the connector is molded over the filament loop portion.

14. The system of claim 11 wherein the structure at the distal end of the needle includes a hook adapted to releasably engage the loop portion of the first or second connector.

15. The system of claim 11 wherein the structure at the distal end of the needle includes a recess adapted to receive the loop portion of the first or second connector.

16. The system of claim 10 wherein the first and second connectors include a ball portion extending from the base portion.

17. The system of claim 16 wherein the structure at the distal end of the needle includes a slot adapted to releasably engage the ball portion of the first or second connector.

18. The system of claim 10 wherein the at least one surgical instrument for implanting the sling includes a helical needle portion adjacent the proximal end of the needle.

19. An implantable sling for treating incontinence comprising
an elongate rectangular mesh sized and shaped to be secured in a tissue pathway of a patient to treat incontinence, the mesh having first and second ends and a middle portion extending therebetween, the first and second ends having a reduced width compared to the middle portion; and
first a second connectors molded over the respective first and second ends, the first and second connectors each having structure for releasable association with a surgical instrument during implantation.

20. The sling of claim 19 wherein the first and second connectors include a base portion and a discrete loop portion, with the base portion being molded over the sling end and the discrete loop portion, and wherein the sling includes at least one sheath enclosing the first and second ends of the mesh, and wherein the first and second connectors are molded over at least one sheath.
